(19) United States
(12) Patent Application Publication
(10) Pub. No.: US 2025/0090016 A1
(43) Pub. Date: Mar. 20, 2025

(54) DEEP LEARNING BASED RETINAL VESSEL PLEXUS DIFFERENTIATION IN OPTICAL COHERENCE TOMOGRAPHY ANGIOGRAPHY

(71) Applicant: Carl Zeiss Meditec, Inc., Dublin, CA (US)

(72) Inventors: Jamie L. Shaffer, Bothell, WA (US); Luis De Sisternes, San Francisco, CA (US); Warren Lewis, Yellow Springs, OH (US); Niranchana Manivannan, Fremont, CA (US); Aaron Y. Lee, Seattle, WA (US)

(73) Assignee: Carl Zeiss Meditec, Inc., Dublin, CA (US)

(21) Appl. No.: 18/887,488

(22) Filed: Sep. 17, 2024

Related U.S. Application Data
(60) Provisional application No. 63/539,528, filed on Sep. 20, 2023.

(51) Int. Cl. A61B 3/10; G06T 7/00

(52) U.S. Cl. CPC A61B 3/102; G06T 7/0012; G06T 2207/10101

(57) ABSTRACT

A system, method and/or device for identifying and categorizing vascular structures in function optical coherence tomography angiography (OCTA) data, without the use of OCT structural data. The method for extracting structural retinal layer information of an eye from OCTA volume data may comprise accessing a plurality of (OCTA) en face images from the OCTA volume data; submitting the en face images to a data processing module configured to identify regions of superficial vascular plexus (SVP) and deep vascular plexus (DVP) within the en face images; and designating at least one identified region of DVP that is superficial to an identified SVP region as a retinal layer.

### CROSS-REFERENCE TO RELATED APPLICATIONS

This application claims priority to, and the benefit of, Provisional Patent Application No. 63/539,528, filed Sep. 20, 2023 and titled “DEEP LEARNING BASED RETINAL VESSEL PLEXUS DIFFERENTIATION IN OPTICAL COHERENCE TOMOGRAPHY ANGIOGRAPHY,” which is incorporated by reference herein in its entirety for all purposes.

### FIELD

The present disclosure is generally directed to retinal vascular identification using optical coherence tomography (OCT). More specifically, it is directed to the identification and categorization of retinal vessel plexuses using OCT angiography data and to the tissues between the superficial vessels and the inner limiting membrane.

### BACKGROUND

Optical coherence tomography (OCT) is a non-invasive imaging technique that uses light waves to penetrate tissue and produce image information (of structures) at different depths within the tissue, such as an eye. Generally, an OCT system is an interferometric imaging system based on detecting the interference of a reference beam and backscattered light from a sample illuminated by an OCT beam. Each scattering profile in the depth direction (e.g., z-axis or axial direction) may be reconstructed individually into an axial scan, or A-scan, that may identify different structural boundaries within the sample encountered by the OCT beam. Cross-sectional slice images (e.g., two-dimensional (2D) bifurcating scans, or B-scans) and volume images (e.g., 3D cube scans, or C-scans) may be built up from multiple A-scans acquired as the OCT beam is scanned/moved through a set of transverse (e.g., x-axis and/or y-axis) locations on the sample. When applied to the retina of an eye, OCT generally provides structural data that, for example, permits one to view, at least in part, distinctive tissue layers and vascular structures of the retina. OCT angiography (OCTA) expands the utility of an OCT system from imaging structures to also identify (e.g., render in image format) functional data, such as the presence, or lack, of blood flow in retinal tissue. For example, OCTA may identify blood flow by identifying differences over time (e.g., contrast differences) in multiple OCT scans of the same retinal region, and designating differences in the scans that meet predefined criteria as blood flow.

An OCT system also permits construction of a planar (2D), frontal view (e.g., en face) image of a select portion of a tissue volume (e.g., a target tissue slab (sub-volume) or target tissue layer(s), such as the retina of an eye). Examples of other 2D representations (e.g., 2D maps) of ophthalmic data provided by an OCT system may include layer thickness maps and retinal curvature maps. For example, to generate layer thickness maps, an OCT system may combine en face images (or slabs), 2D vasculature maps of the retina, with multilayer segmentation data. Thickness maps may be based, at least in part, on measured thickness difference between retinal layer boundaries. Vasculature maps and OCT enface images may be generated, for example, by projecting on to a 2D surface a sub-volume (e.g., tissue slab) defined between two layer-boundaries. The projection may use the sub-volume's mean, sum, percentile, or other data aggregation method. Thus, the creation of these 2D representations of a 3D volume (or sub-volume) data often relies on the effectiveness of automated segmentation algorithms to identify the layers upon which the 2D representations are based. The identified retinal layers may be used to distinguish between different vascular plexuses. Therefore, if the retinal segmentation is incorrect, this can lead to misidentification of vascular plexuses.

Prior art automated segmentation algorithms may suffer from low quality OCT structural data, or pathology that may alter or obscure the shape of a retinal layer from the expected norm. This further complicates the identification of vascular plexuses when using OCT structural data. It would be beneficial to have an automated method for vascular plexus identification that does not rely on OCT structural data, or does not rely solely on OCT structural data.

It is an object of the present disclosure to provide an automated/automatic vascular plexus identification system/method that avoids the use of OCT structural data.

It is another object of the present disclosure to provide an automated/automatic system/method that provide retinal structure information to augment retinal layer data provided by purely OCT structural data.

It is a further object of the present disclosure to provide a method/system of identification of region above (superior) to the superficial plexus and below (inferior) to the inner limiting membrane.

### SUMMARY

The above objects are met in a method/system that uses OCTA data and avoids (or optionally augments) the use of OCT structural data to identify and classify different retinal vascular plexuses. As mentioned above, distinguishing between different vascular plexuses may rely on proper identification of retinal layers (such as provided by OCT data), but it has been found that OCT layer segmentations are not necessarily the correct boundaries even if segmented correctly. That is, vascular plexuses (vessels) may not be strictly confined by these layers. Herein is presented various neural network architectures and data augmentation techniques for defining a machine model that receives OCTA data as input, and outputs identified retinal vessel plexuses and their categorization.

More specifically, the above objects are met in a system/method/apparatus for extracting structural retinal layer information of an eye from optical coherence tomography angiography (OCTA) volume data (e.g., in the absence of OCT structural data), including using a computing device to: access one or more (OCTA) en face images from the OCTA volume data; submit the en face images to a data processing module configured to identify regions of superficial vascular plexus (SVP) and deep vascular plexus (DVP) within the en face images; and designate at least one identified region of DVP that is superficial to an identified SVP region as a retinal layer.

In the present example, the data processing module may include a machine model whose training data set includes a combination of single class images and synthetic 2 class images, where the single class images are comprised of data from only superficial plexuses, deep plexuses, or avascular regions, and the synthetic 2 class images are defined by pairing single class images and their corresponding masks including blending a randomly placed and sized region from an adjacent single class image into each synthetic 2 class image. The data processing module may include a deep learning machine model trained to identify and categorize vasculatures within OCTA data in the absence of OCT structural data.

Optionally, the at least one identified region of DVP that is superficial to an identified SVP region is designated a retinal nerve fiber layer (RNFL). In this case, the region designated as a retinal nerve fiber layer may be incorporated into an (automated) retinal layer segmentation algorithm.

If desired, the upper boundary of the designated RNFL may be defined by the inner limiting membrane (ILM) of the eye.

Furthermore, or optionally, the at least one identified region of DVP may be reclassified as a subdivision of the SVP.

The above objects are also met in a system/apparatus/method for identifying a target tissue region in a volume of retinal tissue of an eye, including using a data processor to: access multiple (OCTA) enface images from OCTA volume data collected from the eye; identify superficial retina vessels within the eye based on the collected enface images; and designate the area between the identified superficial retinal vessels and the inner limiting membrane (ILM) of the eye as the target tissue.

In the present example, identification of the inner limiting membrane may be based on a its definition within an optical coherence tomography (OCT) scan.

Additionally, the data processor may use a machine learning model or conventional image processing to identify the area between the superficial retinal vessel and the inner limiting membrane.

The present example may further include steps for incorporating the identified area between the ILM at the top and the superficial retinal vessels below into an automated retinal layer segmentation algorithm.

Other objects and attainments together with a fuller understanding of the disclosure will become apparent and appreciated by referring to the following description and claims taken in conjunction with the accompanying drawings.

Several publications may be cited or referred to herein to facilitate the understanding of the present disclosure. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings wherein like reference symbols/characters refer to like parts:

FIG. 1 illustrates a process for the construction of training and evaluation datasets. In Part (a), an OCTA volume is pre-processed to create (b) single class slabs containing data from only the superficial plexus, deep plexus, or avascular region, along with a normalized version of each image. These single class images are paired to create (c) synthetic 2-class images and their corresponding masks (note that only the case of a deep region inserted into a superficial image is shown), in accordance with various embodiments. Unlabeled thin slabs (d) are created for qualitative analysis from OCTA volumes in the reserved test set, in accordance with various embodiments.

FIG. 2. illustrates an exemplary process wherein thin slabs from an OCTA cube are split into (a) thin slabs (lightened somewhat for print), plexus predictions (b) on each thin slab are obtained from a trained model, these predictions are reassembled as a volume (c) and then sliced in the B-scan direction to perform qualitative analysis in comparison to flow and structure B-scans of the same B-scan slice (d), in accordance with various embodiments.

FIGS. 3A and 3B show exemplary LinkNet predictions overlaid on flow images (left column) and OCT structure (right column) for 3 OCTA volumes from a reserved test set. The B-scans for Patients #1 and #3 are through the center of the volume; the B-scan for Patient #2 is a peripheral B-scan, in accordance with various embodiments.

FIG. 4 shows Table 1, which is an exemplary table of training, validation, and reserved test groups after splitting by patient, in accordance with various embodiments.

FIG. 5 shows Table 2, which is an exemplary table of Dice scores on reserved test images for two model architectures: UNet with DenseNet backbone, and LinkNet with DenseNet backbone. Training with combined single+synthetic 2-class data provided the best options for both models; LinkNet performed better overall, in accordance with various embodiments.

FIG. 6 illustrates an example of a slit scanning ophthalmic system for imaging a fundus, in accordance with various embodiments.

FIG. 7 illustrates an exemplary generalized frequency domain optical coherence tomography system used to collect 3D image data of the eye suitable for use with the present disclosure, in accordance with various embodiments.

FIG. 8 shows an exemplary OCT B-scan image of a normal retina of a human eye, and illustratively identifies various canonical retinal layers and boundaries, in accordance with various embodiments.

FIG. 9 shows an example of an en face vasculature image, in accordance with various embodiments.

FIG. 10 shows an exemplary B-scan of a vasculature (OCTA) image, in accordance with various embodiments.

FIG. 11 illustrates an example of a multilayer perceptron (MLP) neural network, in accordance with various embodiments.

FIG. 12 shows an exemplary simplified neural network consisting of an input layer, a hidden layer, and an output layer, in accordance with various embodiments.

FIG. 13 illustrates an example convolutional neural network architecture, in accordance with various embodiments.

FIG. 14 illustrates an example U-Net architecture, in accordance with various embodiments.

FIG. 15 illustrates an example computer system (or computing device or computer), in accordance with various embodiments.

### DETAILED DESCRIPTION

Current techniques for segmenting the vascular plexuses of the retina primarily involve a two-step process of first segmenting the structural layers of the retina (e.g., using structural OCT data), and then using the structural boundaries (of the layers) to infer the segmentation boundaries of the vasculature. More specifically, vasculature and vascular plexuses of the retina, which extend through the tissue layers without being strictly bound by these layers, may be segmented by first solving the problem of structural segmentation to identify structural boundaries and then using the structural boundaries as guiding elements for segmentation of the vasculature. While this approach uses structural OCT data that provides structural boundaries that simplify the problem, the use of easily visualized but biologically distinct structural elements to define the vasculature can produce errors and artifacts, particularly in the presence of pathology. Herein it is proposed that the vasculature observed in OCTA can be differentiated by (functional) vascular patterns (e.g., blood flow data) alone, without relying on segmented structural (OCT) data. Additionally, an automated method to divide (or categorize/identify parts of) the vasculature using only OCTA data is presented.

Vascular data from slabs (e.g., 2D frontal views (e.g., along the axial direction), or en face image) of 235 OCTA cubes, or C-scans, (from 33 patients) were processed using handcrafted techniques to ensure only vessels from a single plexus were present. In addition, thin slabs from the volume (C-scan) were generated as unlabeled data for qualitative model evaluation. For example, a thin slab may be defined by a depth (or slab thickness) of 10 or fewer axial pixel sections, depending upon the axial resolution, and may correspond to approximately a 10 μm vertical slice of the volume. Patient-level partitioning was used to separate images into training, validation, and test sets. Data libraries consisted of enface scans of single-class images with (image) augmentation (e.g., flip, rotate, contrast, etc.), and single patient synthetic images created by blending a randomly placed and sized region from an adjacent class into each image. For example, a synthetic 2 class image may be defined by pairing single class images and their corresponding masks, including blending a randomly placed and sized region from an adjacent single class image into each synthetic 2 class image. A U-Net (machine) model with DenseNet 121 backbone (as described more fully below) pretrained on ImageNet was modified to predict only 3 classes and then fine-tuned using various combinations of these data libraries. A LinkNet (machine) model was similarly trained. A general discussion of neural networks, including convolutional neural networks and U-nets, is provided below. As it would be understood by one versed in the art, a DenseNet is a type of convolutional neural network that uses dense connections between layers, through Dense Blocks, where layers (with matching feature-map sizes) are connected with each other. As it is understood in the art, a LinkNet is a light deep neural network architecture designed for performing semantic segmentation, and is typically used for tasks such as self-driving vehicles, augmented reality, etc. Quantitative model evaluation was performed for labeled en face images. Qualitative evaluation was performed on unlabeled en face thin slab images from an OCTA volume and viewed as both en face and B-scan slices. The LinkNet model performed better on single class en face images (Dice 0.99, 0.98, 0.98 for superficial, deep, and avascular classes) and on 2-class images (Dice 0.98, 0.96, 0.95). As it would be understood, a Dice score, or Dice Similarity Coefficient, is a measure of the similarity between two sets of data, with Dice 0 generally indicating no similarity and Dice 1 indicating a 100% similarity. Both the U-net and LinkNet models demonstrated a difficulty with differentiating deep and avascular regions, but this led to an interesting finding. Use of an oversimplification of the tissue into only 3 possible labels caused an interesting mislabeling when the model recognized distinct layers in the portion anterior to the superficial plexus. As is discussed more fully below, the present approach provides a novel approach that facilitates the segmenting of upper (anterior) regions, which was previously known to be challenging.

The retina is a multi-layered tissue in the posterior eye which transduces light into neural signals to process vision. The retinal tissue receives oxygen and nutrients via its blood supply through capillary networks called plexuses that travel through the different retinal layers. Any disease that disrupts the plexuses can lead to retinal dysfunction and affect vision. Optical coherence tomography angiography (OCTA) is a three-dimensional (3D) non-invasive imaging technique that can be used to visualize the retinal vasculature. Early OCTA pathology and imaging studies of the retina identified two separate plexuses within the retina: the superficial and deep plexus. See for example, Snodderly, D. M., et al. “Neural-Vascular Relationships in Central Retina of Macaque Monkeys (Macaca Fascicularis),” The Journal of Neuroscience: The Official Journal of the Society for Neuroscience, vol. 12, no. 4, April 1992, pp. 1169-93; Spaide, Richard F., et al. “Retinal Vascular Layers Imaged by Fluorescein Angiography and Optical Coherence Tomography Angiography.” JAMA Ophthalmology, vol. 133, no. 1, January 2015, pp. 45-50, both of which are herein incorporated in their entirety by reference. With more recent advances in imaging, some authors have further subdivided the superficial and deep plexuses into as many as four separate vascular plexuses (as is described, for example, in Garrity, Sean T., et al. “Quantitative Analysis of Three Distinct Retinal Capillary Plexuses in Healthy Eyes Using Optical Coherence Tomography Angiography.” Investigative Ophthalmology & Visual Science, vol. 58, no. 12, October 2017, pp. 5548-55; Lavia, Carlo, et al. “Retinal Capillary Plexus Pattern and Density from Fovea to Periphery Measured in Healthy Eyes with Swept-Source Optical Coherence Tomography Angiography.” Scientific Reports, vol. 10, no. 1, January 2020, p. 1474; Hormel, Tristan T., et al. “Plexus-Specific Retinal Vascular Anatomy and Pathologies as Seen by Projection-Resolved Optical Coherence Tomographic Angiography.” Progress in Retinal and Eye Research, vol. 80, January 2021, p. 100878; and Campbell, J. P., et al. “Detailed Vascular Anatomy of the Human Retina by Projection-Resolved Optical Coherence Tomography Angiography.” Scientific Reports, vol. 7, February 2017, p. 42201, all of which are herein incorporated in their entirety by reference). However, these divisions still preserve the early distinction between the superficial and deep plexus, as Hormel et al. clarify. Posterior to the deep plexus lie the avascular outer layers of the retina, where the lack of vasculature is thought to help reduce any disruption in the light sensing photoreceptors that are located in this area, as explained in Provis, Jan M., et al. “Adaptation of the Central Retina for High Acuity Vision: Cones, the Fovea and the Avascular Zone.” Progress in Retinal and Eye Research, vol. 35, July 2013, pp. 63-81, herein incorporated in its entirety by reference.

Any retinal disease that affects the vasculature may affect the retinal plexus in a disease dependent manner. Glaucoma, diabetes, hypertension, retinal vascular occlusion, age-related macular degeneration, retinitis pigmentosa, and many other diseases have been associated with varying changes in the plexus layer structure and/or thickness. See, for example, Liu, Liang, et al. “Projection-Resolved Optical Coherence Tomography Angiography of the Peripapillary Retina in Glaucoma.” American Journal of Ophthalmology, vol. 207, November 2019, pp. 99-109; Takusagawa, Hana L., et al. “Projection-Resolved Optical Coherence Tomography Angiography of Macular Retinal Circulation in Glaucoma.” Ophthalmology, vol. 124, no. 11, November 2017, pp. 1589-99; Carnevali, Adriano, et al. “Optical Coherence Tomography Angiography Analysis of Retinal Vascular Plexuses and Choriocapillaris in Patients with Type 1 Diabetes without Diabetic Retinopathy.” Acta Diabetologica, vol. 54, no. 7, July 2017, pp. 695-702; Sun, Christopher, et al. “Systemic Hypertension Associated Retinal Microvascular Changes Can Be Detected with Optical Coherence Tomography Angiography.” Scientific Reports, vol. 10, no. 1, June 2020, p. 9580; Coscas, Florence, et al. “Optical Coherence Tomography Angiography in Retinal Vein Occlusion: Evaluation of Superficial and Deep Capillary Plexa.” American Journal of Ophthalmology, vol. 161, January 2016, pp. 160-71.e1-2; and Koyanagi, Yoshito, et al. “Optical Coherence Tomography Angiography of the Macular Microvasculature Changes in Retinitis Pigmentosa.” Acta Ophthalmologica, vol. 96, no. 1, February 2018, pp. e59-67, all of which are herein incorporated in their entirety by reference. It is noted, however, that the literature is generally limited by small cohort sizes, manually segmented retinal images and conflicting conclusions. Although it is understood that there are microvascular changes in the capillary network that occur in many ophthalmic diseases, it has heretofore not been fully understood what these changes are with respect to the different plexus layers.

To assist in further study of retinal plexuses, previous studies have attempted to algorithmically segment the superficial and deep plexuses. These prior art techniques mostly rely on creating retinal layer segmentation masks from structural optical coherence tomography (OCT) images. With the retinal layers segmented on structural OCT, the algorithms then infer the plexus boundaries on the OCTA images. By contrast, the present disclosure avoids the step of requiring OCT for every volume of OCTA, and using the OCT as boundaries for the plexus. Examples of prior art techniques are provided in Hwang, Thomas S., Ahmed M. Hagag, et al. “Automated Quantification of Nonperfusion Areas in 3 Vascular Plexuses With Optical Coherence Tomography Angiography in Eyes of Patients With Diabetes.” JAMA Ophthalmology, vol. 136, no. 8, August 2018, pp. 929-36, and in Hwang, Thomas S., Miao Zhang, et al. “Visualization of 3 Distinct Retinal Plexuses by Projection-Resolved Optical Coherence Tomography Angiography in Diabetic Retinopathy.” JAMA Ophthalmology, vol. 134, no. 12, December 2016, pp. 1411-19, both herein incorporated in their entirety by reference. A challenge to label or identify these vascular layers is that their boundaries do not follow the retinal layers that are seen on OCT images, as is explained in Campbell, J. P., et al. “Detailed Vascular Anatomy of the Human Retina by Projection-Resolved Optical Coherence Tomography Angiography.” Scientific Reports, vol. 7, February 2017, p. 42201, herein incorporated in its entirety by reference.

To overcome the above mentioned difficulties, here is demonstrated a fully automated segmentation method for identifying the superficial plexus, deep plexus and avascular outer layer of the retina using only OCTA as input (e.g., without the use of OCT structural data). The present method/system/mechanism/procedure has the potential to automatically quantify the retinal vasculature without human intervention or structural OCT input to study microvascular changes in ophthalmologic disease.

### 2. Methods

### 2.1 Data Construction

Vascular data from slabs of 235 OCTA cubes (C-scans) from 33 patients consisting of 6×6 mm (500×500 pixel) enface images x 1536 pixels was obtained using a Plexus Elite 9000 (Carl Zeiss Meditec Inc, Dublin, CA) in association with Bay Area Retina Associates (Walnut Creek, CA). The data includes both healthy and diseased eyes, but the cubes were not labeled to distinguish these. This study used de-identified data to remove any identification linking the data to the patient from which it was obtained. The OCTA data cubes were processed to create several distinct datasets, including single class images, synthetic 2 class images, and unlabeled thin slabs as described below.

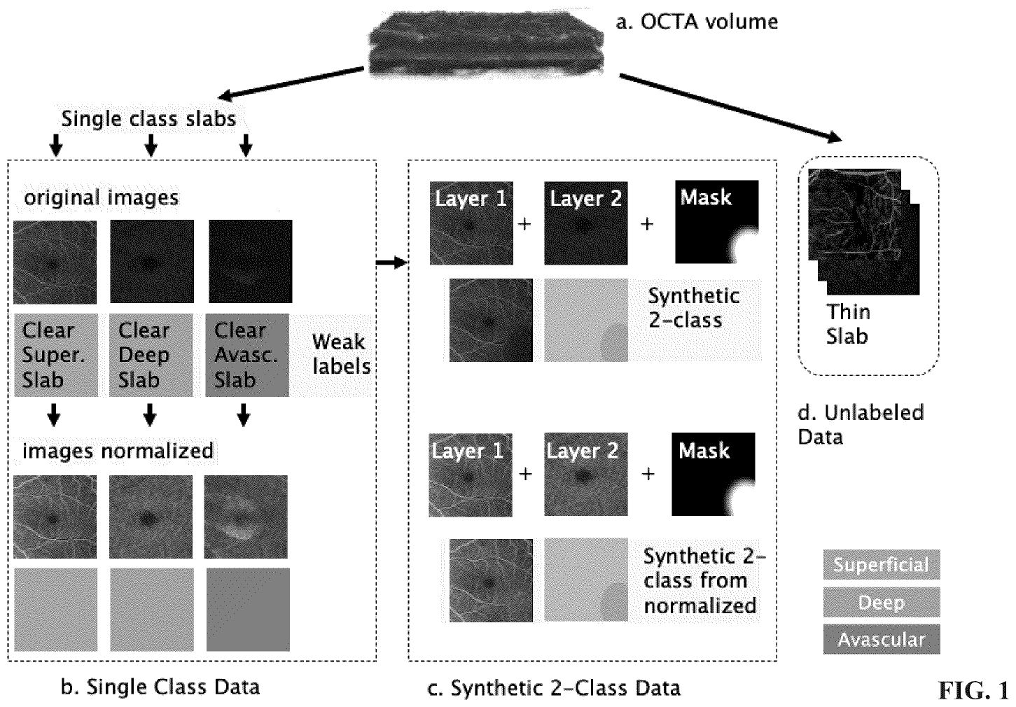

Single class dataset construction. FIG. 1 shows an exemplary process for the construction of training and evaluation datasets. In Part (a), an OCTA volume is pre-processed to create (b) single class slabs containing data from only the superficial plexus, deep plexus, or avascular region, along with a normalized version of each image. These single class images are paired to create (c) synthetic 2-class images and their corresponding masks (note that only the case of a deep region inserted into a superficial image is shown). Unlabeled thin slabs (d) are created for qualitative analysis from OCTA volumes in the reserved test set.

The single class dataset consists of 235 superficial, 235 deep, and 235 avascular en face OCTA slab projections (one set from each of the 235 cubes) and their corresponding weakly labeled single-class masks (FIG. 1 b). OCTA layer segmentation was performed automatically in conjunction with slab-based projection removal; expert review made hand corrections to ensure that three classes (superficial, deep, and avascular) were generated from each cube. This data is therefore regard as weakly labeled data since it has not been explicitly labeled by hand to annotate individual vessel or plexus regions, but instead relies primarily on image-processing techniques and assumptions regarding the data. Structural OCT images recorded simultaneously with the OCTA were used as reference images for the manual corrections of the single class training images and are shown in later figures as reference for the qualitative analysis of the model's predicted class labels. No structural images were used for training or quantitative evaluation. The present embodiment included original images without modification as well as a second set of the same images processed by ignoring the bottom 2% of values, top 1% of values, and renormalizing the image.

Synthetic 2 class dataset construction. Leveraging the single class dataset, a dataset of synthetic 2 class images was created by blending a randomly placed and sized region from an adjacent class of the original image (FIG. 1 c), creating 4 additional images for each of the 235 cubes: blend a small superficial region into a deep image (sD), blend a small deep region in a superficial image (dS), blend a small deep region into an avascular image (dA), and blend a small avascular region into a deep image (aD). For each 2 class pairing, a new grayscale mask was generated to contain an ovoid region with blurred edges to create a blended transition. The ovoid was constrained to ensure that a full transition from 100% majority class to a much smaller region of 100% minority class occurred over the region of blended edges. This blending mask was then applied to the paired images to create the dataset image, and a 50% threshold was applied to the blending mask to create the final 2 class prediction mask. This process was repeated using the normalized versions of the single class images, resulting in a total of 8 additional images for each of the 235 cubes.

Thin slab dataset construction. Thin slabs of unlabeled data (no ground truth) were also created (FIG. 1 d). After applying a volume-based projection removal, 2 dimensional thin slab images were created by averaging 5 axial pixel sections (corresponding to approximately a 10 μm vertical slice of the volume) of data through the retinal area of the OCTA volume. The retinal area was defined with structural borders (e.g., retinal layers) of the internal limiting membrane (ILM) to retinal pigment epithelium (RPE) and the slab contours matched ILM and RPE at the extrema and gradually adjusted from one contour to the other through the subvolume. It is noted that contour matching may be based on the OCT layers described above, but this does not require OCT layer segmentation. In the present example, it was used for the library construction and to narrow the data to the region of interest. No attempt was made to locate or conform with inner plexiform layer and inner nuclear layer junction (IPL/INL) or outer plexiform layer and outer nuclear layer junction (OPL/ONL) boundaries in this subvolume.

Original images (500×500 pixels) in each dataset were converted to 256×256 for training and inference and upscaled to 500×500 with nearest neighbor interpolation for visual analysis.

### 2.2 Dataset Splits for Training, Validation, and Test.

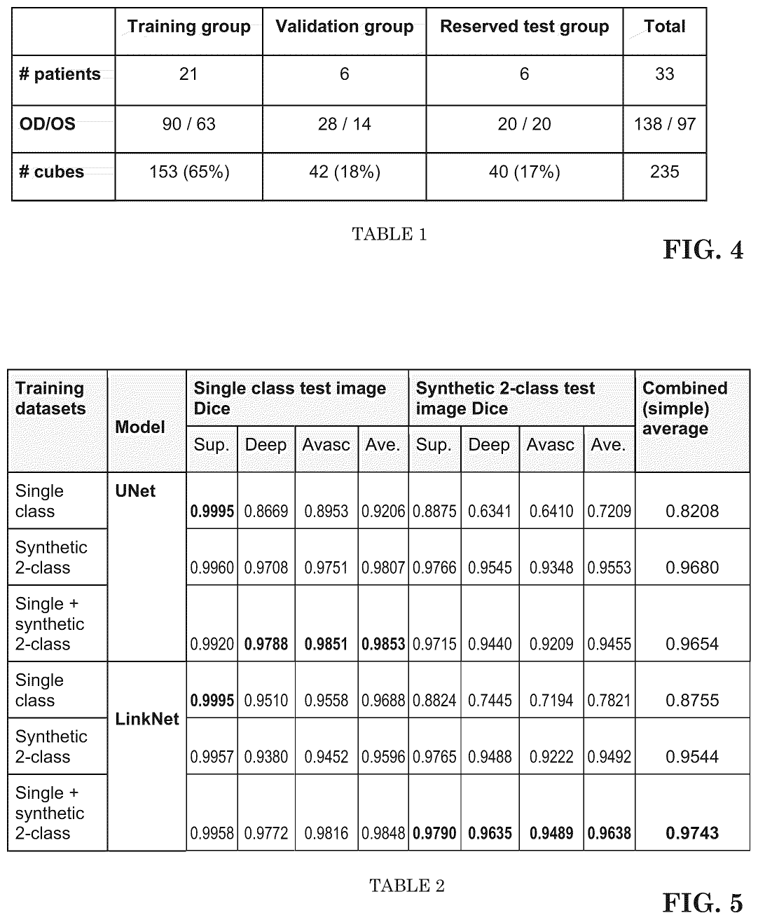

Images in this data set included both healthy and diseased eyes, but this information was not provided with the dataset. Therefore, no attempt was made to balance the train/valid/test sets based on this factor. Instead, manual partitioning without viewing of images was performed, ensuring that each of the 33 patients was represented in only one partition (patient level partitioning), patients with very few image cubes were distributed among the partitions, patients with a large number of image cubes were also distributed among the partitions, and both left and right eyes were represented in each partition. The result was 6 groups: 4 groups ensembled for training, 1 group for validation, and 1 reserved test group. (Table 1. shown in FIG. 4)

### 2.3 Model Architecture and Training

The plexus labeling task that this work addresses falls into the category of semantic segmentation where there have been many successful architectures, including U-Net (for example, as described in Ronneberger et al., “U-Net: Convolutional Networks for Biomedical Image Segmentation”, arXiv, 2015, webpage doi.org/10.48550/ARXIV.1505.04597, herein incorpated in its entirety by reference) and LinkNet (for example, as described in Chaurasia et al., “LinkNet: Exploiting Encoder Representations for Efficient Semantic Segmentation,” 2017 IEEE Visual Communications and Image Processing (VCIP), 2017, pp. 1-4, herein incorporated in its entirety by reference). Inside these models, the structure of the encoder and decoder convolutional layer stacks is termed the backbone of the architecture, and backbones are frequently borrowed from other models, such as VGGNet et al., “Very Deep Convolutional Networks for Large-Scale Image Recognition,” arXiv, 2014, webpage doi.org/10.48550/ARXIV.1409.1556, ResNet (He, Kaiming, et al., “Deep Residual Learning for Image Recognition,” arXiv, 2015, webpage doi.org/10.48550/ARXIV.1512.03385) or DenseNet (Huang, Gao, et al., “Densely Connected Convolutional Networks,” arXiv, 2016, webpage doi.org/10.48550/ARXIV.1608.06993), all of which are herein incorporated in their entirety by reference. In the present embodiment, as an implementation option, the present embodiment used a python library that provides easy access to configurable versions of these architectures and backbones along with pre-trained weights from ImageNet, described in Yakubovskiy, P., “Segmentation Models”, webpage github.com/qubvel/segmentation_models, 2019, incorporated by reference in its entirety.

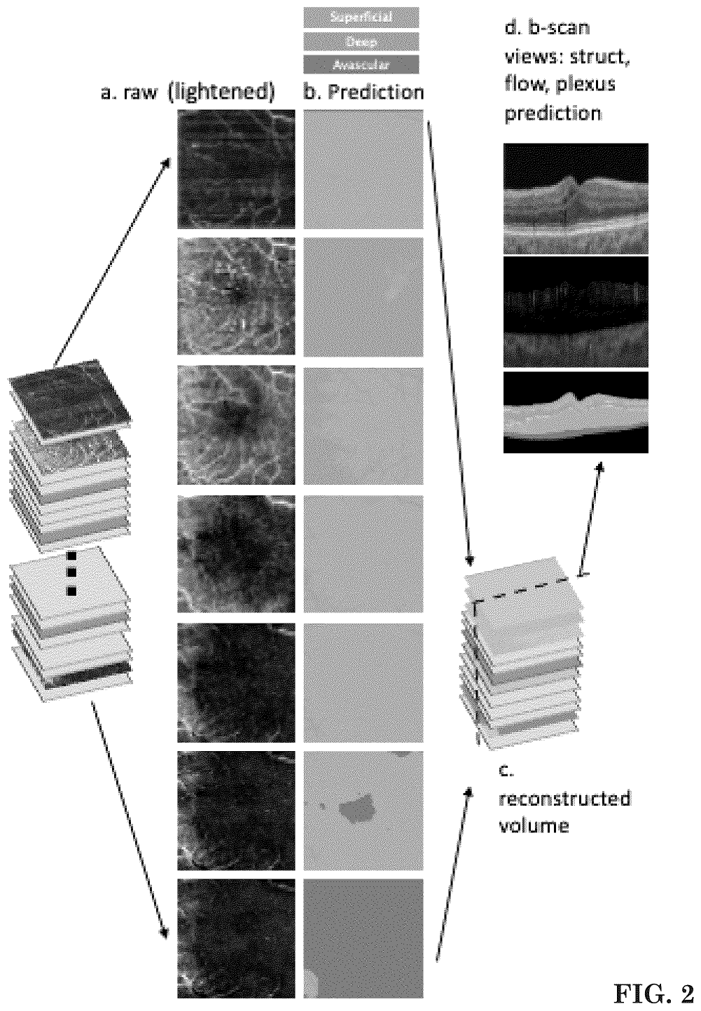

Early experiments with a U-Net model with VGG backbone (K. Simonyan et al.) trained from initial random values provided valuable insight into the challenges of the task: models relied more heavily on pixel values than image features when trained on unnormalized images, training with 2-class images improved Dice scores significantly, training time was lengthy, Dice performance plateaued before achieving reasonable B-scan images, differentiation of the superficial plexus was the easiest task, and predictions through a volume struggled to correctly classify pixels of the superficial retinal layers. These issues were addressed by normalizing images, including the residual 2-class images to provide more multi-class examples, deciding to leave anterior pixel corrections to a post-processing step, and exploring the use of pretrained models and transfer learning. The remainder of this report will describe the results of transfer learning for the two top performing models (FIG. 2): U-Net with DenseNet 121 backbone (hereafter UNet) which has 12,059,641 trainable parameters, and LinkNet with DenseNet 121 backbone (hereafter LinkNet) which at 8,267,417 trainable parameters is 31% smaller than the UNet model.

Both base models (UNet and LinkNet) were initialized with model weights from ImageNet. The classification head was modified to incorporate a softmax activation with 3 output classes. All layers of the encoder were unfrozen (all layers trainable) while setting a maximum learning rate of 0.0001 with the Adam optimizer and leaving other Adam parameters at the default settings; the intuition here was to encourage slower fine-tuning of all layers and discourage completely overwriting the ImageNet weights which might ill-affect the purpose of transfer learning.

The model input was extended by adding a Conv2D (2D convolution) layer to learn 3 filters with kernel size (1,1), thus converting the single channel grayscale input images to the 3 channels expected by the DenseNet backbone. The present loss function was categorical cross-entropy with 3 classes of segmentation.

The ImageNet initialized models were further trained (fine-tuned) on combinations of the raw single class images and synthetic 2-class images. Random image augmentations were used to increase the generalizability of the model and included horizontal flip, vertical flip, and rotation of up to 45 degrees followed by cropping to remove blank regions at the corners of the rotated images. The rotate-and-crop augmentation also introduced a small amount of feature scaling (zoom in) to the center of the image.

The training was performed on servers with NVIDIA CUDA 11.2 and Tesla P100-PCIE-16 GB GPUs. The models were fine-tuned and evaluated on python 3.6.9 with tensorflow 2.3.1, keras 2.4.0, and used the segmentation_models 1.0.1 library, known in the art. Training used a batch size of 32 images.

### Results

All models were trained for 2400 epochs. Training on the single class images took 20 hours, synthetic 2-class images took 30 hours, and training with both datasets took 51 hours.

### Quantitative Evaluation.

Each of the trained models was evaluated on the single class and synthetic 2-class reserved test images separately. The Dice scores were averaged to perform an initial ranking of model performance (Table 2 shown in FIG. 5). It was found that the UNet model performed slightly better when scored on the single class images (averaged Dice 0.9853) while the LinkNet model performed slightly better on the synthetic 2-class images (averaged Dice 0.9638).

### Qualitative Evaluation.

For this evaluation, 500×500 pixel enface thin slabs were extracted from the OCTA cube as described earlier, downsampled to 256×256 pixels, and the models were run on the thin slabs (FIG. 2 a) to acquire the plexus predictions (FIG. 2 b). The predicted masks were then upsampled in a post-processing step to create 500×500 pixel masks. These prediction masks were mapped back onto the original OCTA thin slab regions throughout the volume to create a prediction cube (FIG. 2 c); for voxels included in more than one thin slab, and the most likely class based on the combined predictions was assigned. The prediction cube was vertically sliced to observe the prediction as a B-scan view (FIG. 2 d) and the predictions outside the region of interest (e.g., in the vitreous region) were cropped from the final plots.

Predictions from all 6 models were made on en face thin slabs of an OCTA cube from the reserved test set and reassembled into 6 prediction cubes. The B-scan slices were taken from the unlabeled OCTA cube (flow) and from the OCT cube (structural) to serve as references for qualitative comparison, and the predictions from the top performing LinkNet model were overlaid on these reference images.

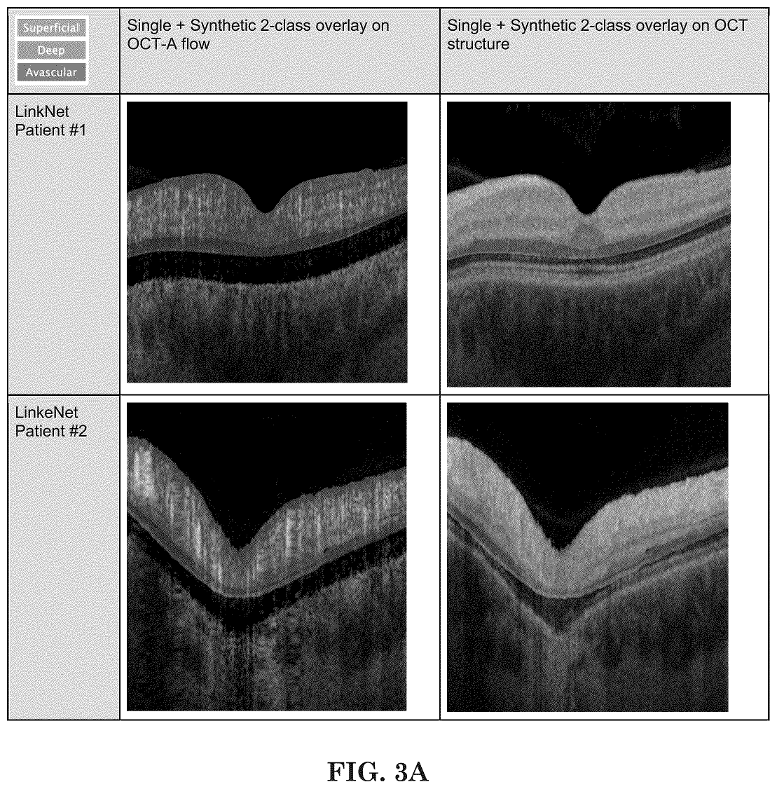

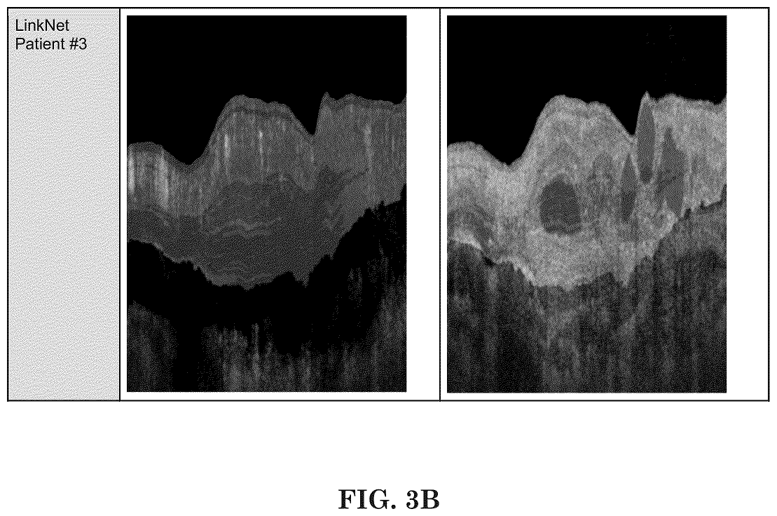

FIGS. 3A and 3B show LinkNet predictions for three patients (on three rows labeled Patient #1, Patient #2, and Patient #3) overlaid on flow images (left column) and OCT structure (right column) for 3 OCTA volumes from a reserved test set. The B-scans for Patients #1 and #3 are through the center of the volume; the B-scan for Patient #2 is a peripheral B-scan. One of the limitations of working with thin slabs is that mapping the en face predictions on the thin slabs back into the cube volume does result in some overlap of prediction masks; these prediction artifacts are on the order of a single line of pixels and have been mapped to the nearest class in the reported images.

When the predictions are overlaid on the OCTA (flow) images and the OCT (structural) images, the advantage of utilizing only OCTA data is more clearly seen. Specifically, while the labels available to the model are limited (superficial, deep, or avascular in this implementation), one can see that the model is differentiating an additional plexus layer superficial to the superficial vascular plexus. This additional layer, which is misclassified as deep vascular plexus, lies along the structural boundary of the retinal nerve fiber layer (RNFL). Previous authors have described a plexus that lies in this anatomic region but have disagreed on the nomenclatures. The previous names include: nerve fiber layer plexus, ganglion cell layer plexus, and radial peripapillary capillary plexus (as describe in Hormel et al.). This is an achievement that is enabled by using only the OCTA data and not presuming that the superficial vascular plexus extends through the full retinal volume in the anterior direction. Additional work is focusing on the refinement of the ability of this model to differentiate this region.

### Discussion

Herein is presented a method to train a deep learning model in the absence of structural OCT data to segment the superficial vascular plexus, deep vascular plexus and avascular layer of the retina using only OCTA data as input. With 235 OCTA cubes from 33 patients for training, test and validation, a Dice score less than 0.95 was achieved on a multi-class segmentation task using a mixed training set of labeled and synthetic data from handcrafted ground-truth masks.

Imaging studies have shown that the retinal plexuses do not travel consistently through all the retinal layers (Campbell et al.), and previously used rules of defining plexus boundaries from structural OCT segmentation boundaries are likely to be inconsistent throughout the whole retina if they follow absolute rules regarding plexus definition in relation to retinal layers. This may partially explain the finding of Spaide, Richard F., and Christine A. Curcio. “Evaluation of Segmentation of the Superficial and Deep Vascular Layers of the Retina by Optical Coherence Tomography Angiography Instruments in Normal Eyes,” JAMA Ophthalmology, vol. 135, no. 3, March 2017, pp. 259, herein incorporated in its entirety by reference, which showed that OCT instrument embedded plexus segmentation models in healthy eyes are inconsistent with their quality of segmentation especially near the center of the eye (fovea), where the plexuses start to merge on top of each other. This degradation in performance is likely because all previous published plexus segmentation algorithms relied on structural OCT segmentations to infer retinal plexus boundaries (as described, for example, in Hwang, Thomas S., Ahmed M. Hagag, et al.; Guo, Yukun, et al. “Automated Segmentation of Retinal Layer Boundaries and Capillary Plexuses in Wide-Field Optical Coherence Tomographic Angiography.” Biomedical Optics Express, vol. 9, no. 9, September 2018, pp. 4429-42; Li, Ang, et al. “Automated Segmentation and Quantification of OCT Angiography for Tracking Angiogenesis Progression.” Biomedical Optics Express, vol. 8, no. 12, December 2017, pp. 5604-16; and Zhu, Qiujian, et al. “A New Approach for the Segmentation of Three Distinct Retinal Capillary Plexuses Using Optical Coherence Tomography Angiography.” Translational Vision Science & Technology, vol. 8, no. 3, 2019, p. 57, https://doi.org/10.1167/tvst.8.3.57). If a patient has specific retinal pathology that affects the segmentation of the retinal layers on structural OCT, this will impede any of the aforementioned plexus segmentation models that use structural OCT. As shown in FIG. 3A, patent #2, the plexus segmentation is overlaid on a structural OCT scan of the peripheral retina. In this location the retinal layers are poorly defined, thus any automatic segmentation of retinal layers on structural OCT would fail. Qualitatively, however, the present network is able to identify the avascular, deep and superficial layers of the vasculature. This same behavior is seen in FIG. 3B, patient #3, where there is significant retinal pathology which makes prior art automatic segmentation algorithms likely to fail. By contrast, the present method is able to grossly assign plexus layers in the correct anatomical hierarchy. It is put forth that the OCTA signal may be preserved in retinal disease that is independent of the vasculature, thus allowing for plexus segmentation without adequate structural OCT layer segmentation.

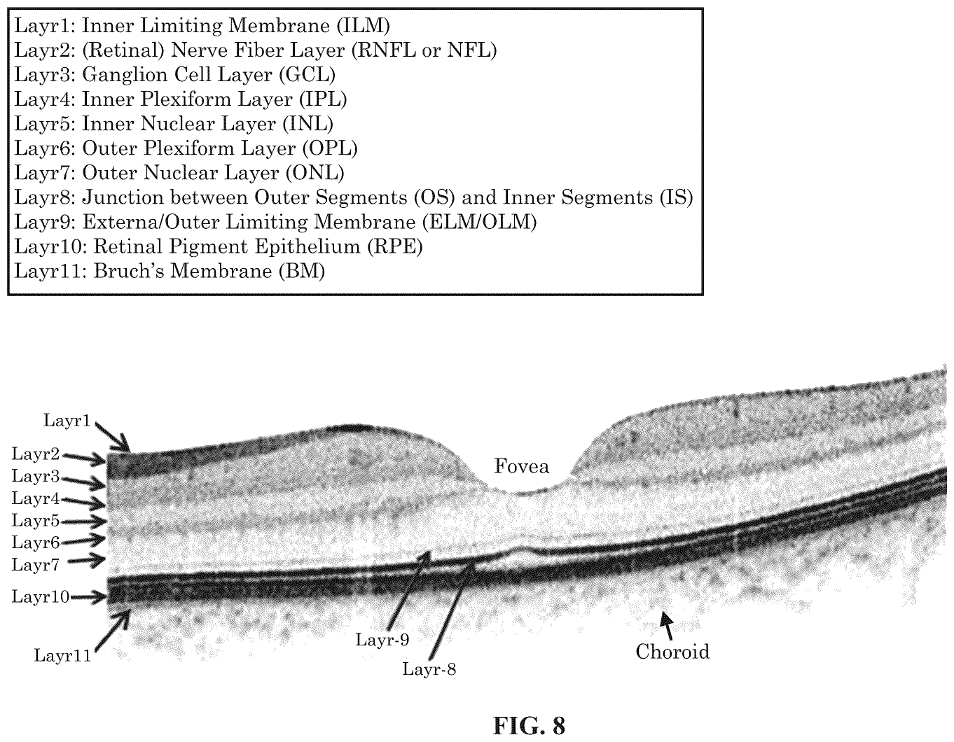

Qualitative evaluation of the segmentation outputs show that the model's performance varies depending on the data it was trained on. The models achieved the highest Dice score and best subjective qualitative evaluation when trained on the combined single class and synthetic 2 class image dataset. Generally, there are two main vascular plexus, a first vascular plexus (e.g., superficial vascular plexus, SVP) at a first depth and a second vascular plexus (e.g., deep vascular complex, DVC) at a second depth deeper than the first depth. The deep vascular complex (DVC) may include the intermediate capillary plexus (ICP) and/or the deep capillary plexus (DCP). Interestingly, as seen in FIG. 3, the model predicts a layer of deep vascular plexus (DVP) superficial to the superficial vascular plexus (SVP). It is noted that the single class predictions did not have this layer, so it could be that it was due to the synthetic data creation and training process. This initially appeared to be a mislabeling by the model, which could be post-processed from the final prediction maps. However, after superimposing the structural OCT B-scan with the OCTA plexus segmentation mask, the misclassified DVP layer lies over the retinal nerve fiber layer (RNFL) on OCT, whose upper boundary is generally defined by the inner limiting membrane (ILM), see FIG. 8. It has been suggested that there is an additional unique plexus which exists as a subdivision of the superficial vascular plexus (SVP) that is defined by its anatomical location within the RFNL and its unique morphology with linearly oriented vasculature extending from the peripapillary space. It is herein put forth that the model's initially supposed mislabeling of a superficial DVP layer on the most superficial layer of the retina (i.e., the RNFL) is because the model is identifying subdivisions of the SVP. That is, the present model (which may be embodied in a method, system, or apparatus) is effective not only for identifying subdivision of the superficial vascular plexus (SVP) that lie above the SVP, but also effective identifying the region of tissue in a volume (e.g., C-scan) of retinal tissue between the superficial retinal vessels (e.g., the superficial vascular plexus, SVP) and the inner limiting membrane (ILM) by using the vasculatures identified using OCTA data combined with a definition of the inner limiting membrane (ILM) from an OCT scan. This can be achieved either by machine learning or by conventional image processing to find the region between the vessel plexus (e.g., SVP) and the ILM. This boundary information (e.g., ILM at top and superiors vessels (e.g., SVP) below) could be used to augment/supplement existing (e.g., automated) retinal layer segmentation techniques or algorithms.

Deep learning models generally perform their task according to the labeled data that is inputted. One or more embodiment of the present disclosure chose to use the superficial vascular plexus and the deep vascular plexus because they represent the two larger networks upon which there is general consensus. Regarding the intermediate capillary plexus, nerve fiber layer plexus, ganglion cell layer plexus, radial peripapillary capillary plexus are smaller subdivision of capillary networks described previously, however, authors have disagreement on the nomenclature and boundaries (Hormel et al.). One or more embodiment of the present disclosure optionally elected to not subdivide the SVP and DVP into these subdivisions because of the lack of consensus on their definitions and locations. Additionally, it was elected not to segment the foveal avascular zone in the manually curated ground truth labels because it is extremely difficult to identify the boundary of where this begins in OCTA (e.g., in OCTA images/data). Optionally, a model in accord with the present disclosure could be trained with additional plexus layers and foveal avascular zone labels and likely achieve similar results, since the present disclosure shows that OCTA alone is sufficient for segmenting retinal plexus layers.

A future application for this kind of model may include fine tuning the layer segmentation in an OCTA volume after rough boundaries have been defined using more conventional methods. For example, when determining the boundary between the superficial and deep capillary plexuses, the existence of larger vessels in the superficial vasculature may produce local deformations of this boundary that may be difficult to follow. A method in accord with the present disclosure makes it possible to adjust the segmentation boundary to be more faithful to the anatomy.

While the model has achieved high Dice scores (>0.95), improvements may be obtained by implementing optional steps. For example, the large vessel tails (artifacts) may be handled within the model prior to training or in a post-processing step. In some images, the presence of the large vessel in the thin slabs deeper in the OCTA cube may extend the superficial plexus somewhat deeper than expected.

Generation of thin slabs that overlap with thin slabs above and/or below by a few pixels can lead to a better solution through multiple evaluations of different instances surrounding the data. A difficulty may be that the generation of these thin slabs may be hit-or-miss and limit the ability of the algorithm to learn from other sets of data or to generalize in some cases of pathology.

Recent work (Hormel et al. 2021) suggests that 4 distinct plexuses may be identified, including the intermediate capillary plexus (ICP) and a plexus lying along the RNFL). The present disclosure used a simplified method with only weak labels of the superficial and deep plexuses in OCTA data. This avoided the difficulty that these plexus are not easily separated by slabs since they may not spread perfectly horizontally and the retina is very thin.

The simplified en face datasets used herein did not identify the foveal avascular zone (FAZ) and therefore trained the model to include this region with the superficial or deep plexus. The FAZ is not found in all patients, and while it was likely identifiable in the present dataset, the present embodiment(s) elected to explore the progress that could be made using the overly simplified classes and weak labels without delineating the FAZ. As a result, the present model does not commit the error of assuming layers are of uniform thickness near the fovea (such as discussed in Spaide and Curcio).

In one or more of the above embodiment(s), a deep-learning model is trained to segment the superficial plexus, deep plexus and avascular layers of the retina using only OCTA as input. By augmenting the training set with synthetically generated 2-class images from adjacent layer classes, the model's performance was improved without requiring multi-class manually segmented images. This method is useful, for example, if structural OCT data is unavailable or if segmentation of OCT data is unreliable due to retinal disease.

Hereinafter is provided a description of various hardware and architectures suitable for the present disclosure.

### Fundus Imaging System

Two categories of imaging systems used to image the fundus are flood illumination imaging systems (or flood illumination imagers) and scan illumination imaging systems (or scan imagers). Flood illumination imagers flood with light an entire field of view (FOV) of interest of a specimen at the same time, such as by use of a flash lamp, and capture a full-frame image of the specimen (e.g., the fundus) with a full-frame camera (e.g., a camera having a two-dimensional (2D) photo sensor array of sufficient size to capture the desired FOV, as a whole). For example, a flood illumination fundus imager would flood the fundus of an eye with light, and capture a full-frame image of the fundus in a single image capture sequence of the camera. A scan imager provides a scan beam that is scanned across a subject, e.g., an eye, and the scan beam is imaged at different scan positions as it is scanned across the subject creating a series of image-segments that may be reconstructed, e.g., montaged, to create a composite image of the desired FOV. The scan beam could be a point, a line, or a two-dimensional area such a slit or broad line. Examples of fundus imagers are provided in U.S. Pat. Nos. 8,967,806 and 8,998,411.

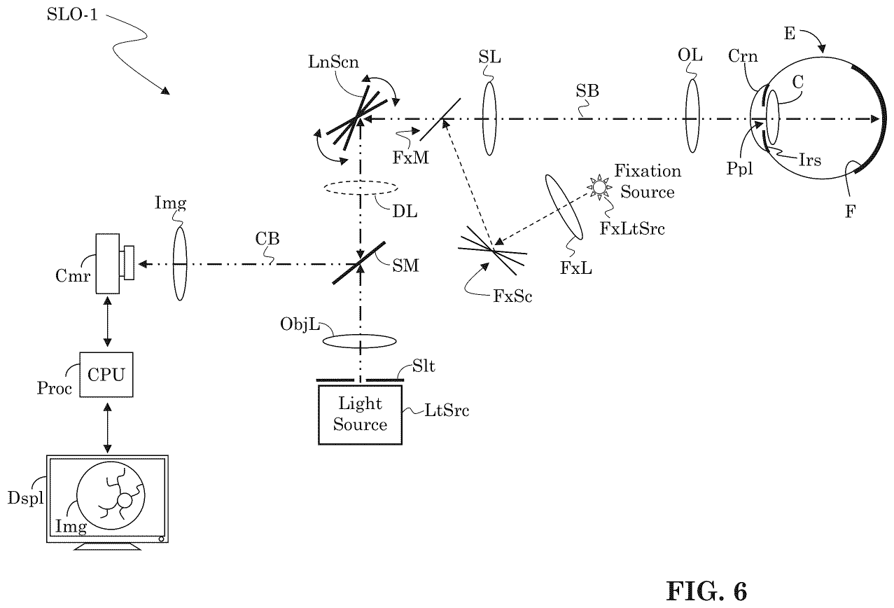

FIG. 6 illustrates an example of a slit scanning ophthalmic system SLO-1 for imaging a fundus F, which is the interior surface of an eye E opposite the eye lens (or crystalline lens) CL and may include the retina, optic disc, macula, fovea, and posterior pole. In the present example, the imaging system is in a so-called “scan-descan” configuration, wherein a scanning line beam SB traverses the optical components of the eye E (including the cornea Crn, iris Irs, pupil Ppl, and crystalline lens CL) to be scanned across the fundus F. In the case of a flood fundus imager, no scanner is used, and the light is applied across the entire, desired field of view (FOV) at once. Other scanning configurations are known in the art, and the specific scanning configuration is not critical to the present disclosure. As depicted, the imaging system includes one or more light sources LtSrc, preferably a multi-color LED system or a laser system in which the etendue has been suitably adjusted. An optional slit Slt (adjustable or static) is positioned in front of the light source LtSrc and may be used to adjust the width of the scanning line beam SB. Additionally, slit Slt may remain static during imaging or may be adjusted to different widths to allow for different confocality levels and different applications either for a particular scan or during the scan for use in suppressing reflexes. An optional objective lens ObjL may be placed in front of the slit Slt. The objective lens ObjL can be any one of state-of-the-art lenses including but not limited to refractive, diffractive, reflective, or hybrid lenses/systems. The light from slit Slt passes through a pupil splitting mirror SM and is directed towards a scanner LnScn. It is desirable to bring the scanning plane and the pupil plane as near together as possible to reduce vignetting in the system. Optional optics DL may be included to manipulate the optical distance between the images of the two components. Pupil splitting mirror SM may pass an illumination beam from light source LtSrc to scanner LnScn, and reflect a detection beam from scanner LnScn (e.g., reflected light returning from eye E) toward a camera Cmr. A task of the pupil splitting mirror SM is to split the illumination and detection beams and to aid in the suppression of system reflexes. The scanner LnScn could be a rotating galvo scanner or other types of scanners (e.g., piezo or voice coil, micro-electromechanical system (MEMS) scanners, electro-optical deflectors, and/or rotating polygon scanners). Depending on whether the pupil splitting is done before or after the scanner LnScn, the scanning could be broken into two steps wherein one scanner is in an illumination path and a separate scanner is in a detection path. Specific pupil splitting arrangements are described in detail in U.S. Pat. No. 9,456,746, which is herein incorporated in its entirety by reference.

From the scanner LnScn, the illumination beam passes through one or more optics, in this case a scanning lens SL and an ophthalmic or ocular lens OL, that allow for the pupil of the eye E to be imaged to an image pupil of the system. Generally, the scan lens SL receives a scanning illumination beam from the scanner LnScn at any of multiple scan angles (incident angles), and produces scanning line beam SB with a substantially flat surface focal plane (e.g., a collimated light path). Ophthalmic lens OL may then focus the scanning line beam SB onto an object to be imaged. In the present example, ophthalmic lens OL focuses the scanning line beam SB onto the fundus F (or retina) of eye E to image the fundus. In this manner, scanning line beam SB creates a traversing scan line that travels across the fundus F. One possible configuration for these optics is a Kepler type telescope wherein the distance between the two lenses is selected to create an approximately telecentric intermediate fundus image (4-f configuration). The ophthalmic lens OL could be a single lens, an achromatic lens, or an arrangement of different lenses. All lenses could be refractive, diffractive, reflective or hybrid as known to one skilled in the art. The focal length(s) of the ophthalmic lens OL, scan lens SL and the size and/or form of the pupil splitting mirror SM and scanner LnScn could be different depending on the desired field of view (FOV), and so an arrangement in which multiple components can be switched in and out of the beam path, for example by using a flip in optic, a motorized wheel, or a detachable optical element, depending on the field of view can be envisioned. Since the field of view change results in a different beam size on the pupil, the pupil splitting can also be changed in conjunction with the change to the FOV. For example, a 45° to 60° field of view is a typical, or standard, FOV for fundus cameras. Higher fields of view, e.g., a widefield FOV, of 60°-120°, or more, may also be feasible. A widefield FOV may be desired for a combination of the Broad-Line Fundus Imager (BLFI) with another imaging modalities such as optical coherence tomography (OCT). The upper limit for the field of view may be determined by the accessible working distance in combination with the physiological conditions around the human eye. Because a typical human retina has a FOV of 140° horizontal and 80°-100° vertical, it may be desirable to have an asymmetrical field of view for the highest possible FOV on the system.

The scanning line beam SB passes through the pupil Ppl of the eye E and is directed towards the retinal, or fundus, surface F. The scanner LnScn1 adjusts the location of the light on the retina, or fundus, F such that a range of transverse locations on the eye E are illuminated. Reflected or scattered light (or emitted light in the case of fluorescence imaging) is directed back along as similar path as the illumination to define a collection beam CB on a detection path to camera Cmr.

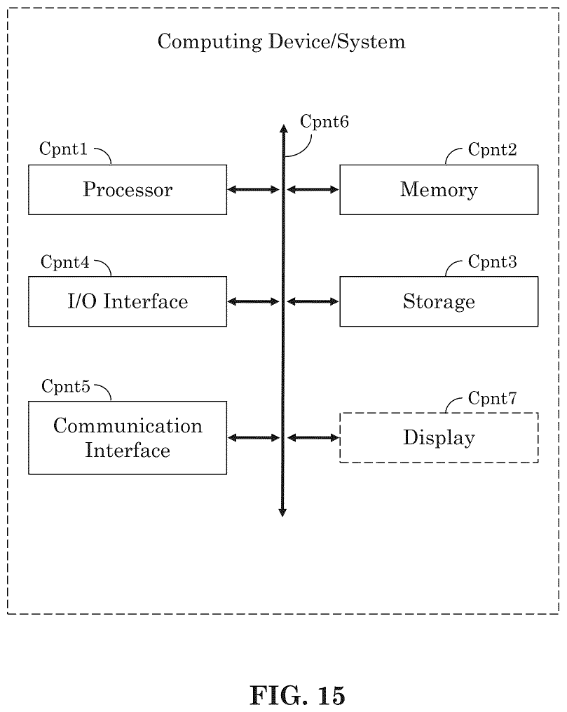

In the “scan-descan” configuration of the present, exemplary slit scanning ophthalmic system SLO-1, light returning from the eye E is “descanned” by scanner LnScn on its way to pupil splitting mirror SM. That is, scanner LnScn scans the illumination beam from pupil splitting mirror SM to define the scanning illumination beam SB across eye E, but since scanner LnScn also receives returning light from eye E at the same scan position, scanner LnScn has the effect of descanning the returning light (e.g., cancelling the scanning action) to define a non-scanning (e.g., steady or stationary) collection beam from scanner LnScn to pupil splitting mirror SM, which folds the collection beam toward camera Cmr. At the pupil splitting mirror SM, the reflected light (or emitted light in the case of fluorescence imaging) is separated from the illumination light onto the detection path directed towards camera Cmr, which may be a digital camera having a photo sensor to capture an image. An imaging (e.g., objective) lens ImgL may be positioned in the detection path to image the fundus to the camera Cmr. As is the case for objective lens ObjL, imaging lens ImgL may be any type of lens known in the art (e.g., refractive, diffractive, reflective or hybrid lens). Additional operational details, in particular, ways to reduce artifacts in images, are described in PCT Publication No. WO2016/124644, the contents of which are herein incorporated in their entirety by reference. The camera Cmr captures the received image, e.g., it creates an image file, which can be further processed by one or more (electronic) data processors or computing devices (e.g., the computer system of FIG. 15). Thus, the collection beam (returning from all scan positions of the scanning line beam SB) is collected by the camera Cmr, and a full-frame image Img may be constructed from a composite of the individually captured collection beams, such as by montaging. However, other scanning configuration are also contemplated, including ones where the illumination beam is scanned across the eye E and the collection beam is scanned across a photo sensor array of the camera. PCT Publication WO 2012/059236 and US Patent Publication No. 2015/0131050, herein incorporated by reference, describe several embodiments of slit scanning ophthalmoscopes including various designs where the returning light is swept across the camera's photo sensor array and where the returning light is not swept across the camera's photo sensor array.

In the present example, the camera Cmr is connected to a processor (e.g., data processing module) Proc and a display (e.g., displaying module, computer screen, electronic screen, etc.) Dspl, both of which can be part of the image system itself, or may be part of separate, dedicated processing and/or displaying unit(s), such as a computer system wherein data is passed from the camera Cmr to the computer system over a cable or computer network including wireless networks. The display and processor can be an all in one unit. The display can be a traditional electronic display/screen or of the touch screen type and can include a user interface for displaying information to and receiving information from an instrument operator, or user. The user can interact with the display using any type of user input device as known in the art including, but not limited to, mouse, knobs, buttons, pointer, and touch screen.

It may be desirable for a patient's gaze to remain fixed while imaging is carried out. One way to achieve this is to provide a fixation target that the patient can be directed to stare at. Fixation targets can be internal or external to the instrument depending on what area of the eye is to be imaged. One embodiment of an internal fixation target is shown in FIG. 6. In addition to the primary light source LtSrc used for imaging, a second optional light source FxLtSrc, such as one or more LEDs, can be positioned such that a light pattern is imaged to the retina using lens FxL, scanning element FxScn and reflector/mirror FxM. Fixation scanner FxScn can move the position of the light pattern and reflector FxM directs the light pattern from fixation scanner FxScn to the fundus F of eye E. Preferably, fixation scanner FxScn is position such that it is located at the pupil plane of the system so that the light pattern on the retina/fundus can be moved depending on the desired fixation location.

Slit-scanning ophthalmoscope systems are capable of operating in different imaging modes depending on the light source and wavelength selective filtering elements employed. True color reflectance imaging (imaging similar to that observed by the clinician when examining the eye using a hand-held or slit lamp ophthalmoscope) can be achieved when imaging the eye with a sequence of colored LEDs (red, blue, and green). Images of each color can be built up in steps with each LED turned on at each scanning position or each color image can be taken in its entirety separately. The three, color images can be combined to display the true color image, or they can be displayed individually to highlight different features of the retina. The red channel best highlights the choroid, the green channel highlights the retina, and the blue channel highlights the anterior retinal layers. Additionally, light at specific frequencies (e.g., individual colored LEDs or lasers) can be used to excite different fluorophores in the eye (e.g., autofluorescence) and the resulting fluorescence can be detected by filtering out the excitation wavelength.

The fundus imaging system can also provide an infrared reflectance image, such as by using an infrared laser (or other infrared light source). The infrared (IR) mode is advantageous in that the eye is not sensitive to the IR wavelengths. This may permit a user to continuously take images without disturbing the eye (e.g., in a preview/alignment mode) to aid the user during alignment of the instrument. Also, the IR wavelengths have increased penetration through tissue and may provide improved visualization of choroidal structures. In addition, fluorescein angiography (FA) and indocyanine green (ICG) angiography imaging can be accomplished by collecting images after a fluorescent dye has been injected into the subject's bloodstream. For example, in FA (and/or ICG) a series of time-lapse images may be captured after injecting a light-reactive dye (e.g., fluorescent dye) into a subject's bloodstream. It is noted that care should be taken since the fluorescent dye may lead to a life-threatening allergic reaction in a portion of the population. High contrast, greyscale images are captured using specific light frequencies selected to excite the dye. As the dye flows through the eye, various portions of the eye are made to glow brightly (e.g., fluoresce), making it possible to discern the progress of the dye, and hence the blood flow, through the eye.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

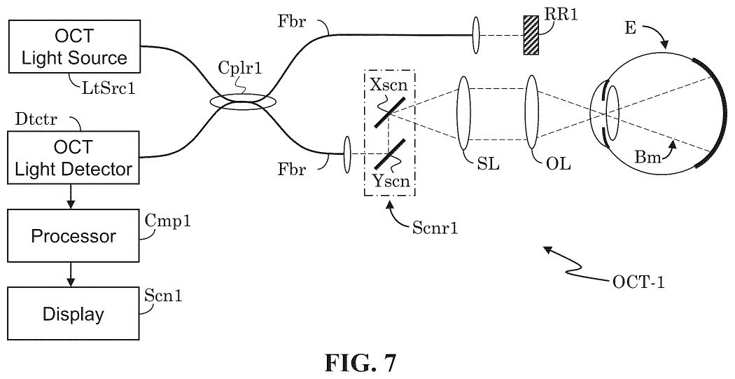

FIG. 7 illustrates a generalized frequency domain optical coherence tomography (FD-OCT) system used to collect 3D image data of the eye suitable for use with the present disclosure. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produce substantially collimated light, and ophthalmic lens OL may then focus onto the sample. The present example illustrates a scan beam that may be scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner may be used to scan the line-field beam across the sample to span the desired FOV. If the scan beam were a full-field beam (e.g., a full-field OCT), no scanner may be used, and the full-field light beam may be applied across the entire, desired FOV at once.

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 15. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13): 2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm may have a tunable optical delay to generate interference. Balanced detection systems are typically used in TD-OCT and SS-OCT systems, while spectrometers are used at the detection port for SD-OCT systems. The disclosure could be applied to any type of OCT system. Various aspects of the disclosure could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiϕ. The absolute value of this complex OCT signal, |Aj|, reveals the profile of scattering intensities at different path lengths, and therefore scattering as a function of depth (z-direction) in the sample. Similarly, the phase, fj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 8. An OCT B-scan of the retinal provides a view of the structure of retinal tissue. For illustration purposes, FIG. 8 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography (OCTA), or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An enface image (sometime termed a “slab”) is a 2D projection of 3D data (e.g., by averaging the intensity of each individual A-scan, such that each A-scan defines a pixel in the 2D projection). For example, an en face OCT image, or slab, may be created from 3D OCT data whose depth range (e.g., thickness or thinness of the slab) is defined as being between two selected depth boundaries, or between two selected tissue layers (e.g., two retinal layers), etc. Similarly, an enface vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

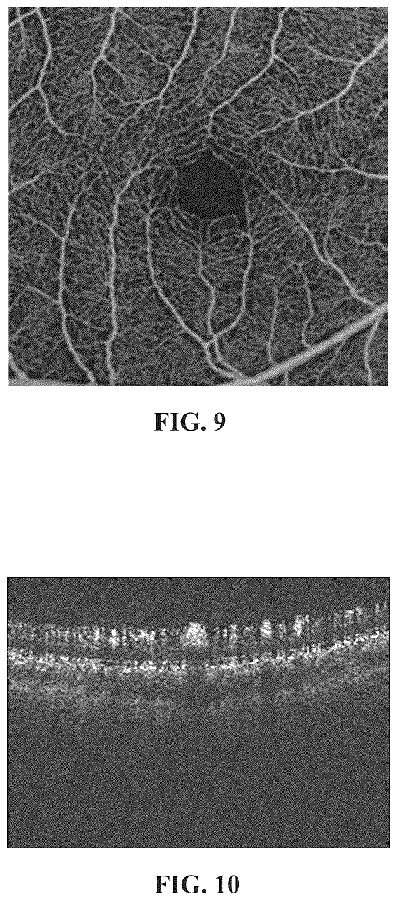

FIG. 9 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the enface (e.g., frontal view) image of the vasculature. FIG. 10 shows an exemplary B-scan of a vasculature (OCTA) image. As illustrated, structural information may not be well-defined since blood flow may traverse multiple retinal layers making them less defined than in a structural OCT B-scan, as shown in FIG. 8. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique involves the injection of a dye to observe vascular flow in the retina. Additionally, in dry age-related macular degeneration, OCTA has been used to monitor a general decrease in choriocapillaris flow. Similarly in wet age-related macular degeneration, OCTA can provide a qualitative and quantitative analysis of choroidal neovascular membranes. OCTA has also been used to study vascular occlusions, e.g., evaluation of nonperfused areas and the integrity of superficial and deep plexus.

### Neural Networks

As discussed above, the present disclosure may use a neural network (NN) machine learning (ML) model. For the sake of completeness, a general discussion of neural networks is provided herein. The present disclosure may use any, singularly or in combination, of the below described neural network architecture(s). A neural network, or neural net, is a (nodal) network of interconnected neurons, where each neuron represents a node in the network. Groups of neurons may be arranged in layers, with the outputs of one layer feeding forward to a next layer in a multilayer perceptron (MLP) arrangement. MLP may be understood to be a feedforward neural network model that maps a set of input data onto a set of output data.

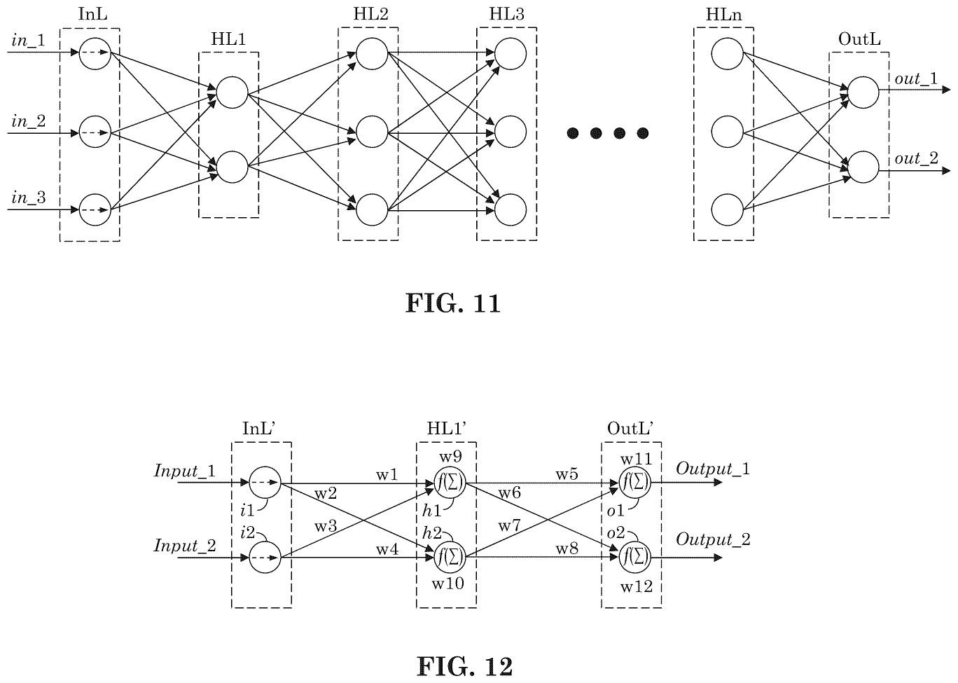

FIG. 11 illustrates an example of a multilayer perceptron (MLP) neural network. Its structure may include multiple hidden (e.g., internal) layers HL1 to HLn that map an input layer InL (that receives a set of inputs (or vector input) in_1 to in_3) to an output layer OutL that produces a set of outputs (or vector output), e.g., out_1 and out_2. Each layer may have any given number of nodes, which are herein illustratively shown as circles within each layer. In the present example, the first hidden layer HL1 has two nodes, while hidden layers HL2, HL3, and HLn each have three nodes. Generally, the deeper the MLP (e.g., the greater the number of hidden layers in the MLP), the greater its capacity to learn. The input layer InL receives a vector input (illustratively shown as a three-dimensional vector consisting of in_1, in_2 and in_3), and may apply the received vector input to the first hidden layer HL1 in the sequence of hidden layers. An output layer OutL receives the output from the last hidden layer, e.g., HLn, in the multilayer model, processes its inputs, and produces a vector output result (illustratively shown as a two-dimensional vector consisting of out_1 and out_2).

Typically, each neuron (or node) produces a single output that is fed forward to neurons in the layer immediately following it. But each neuron in a hidden layer may receive multiple inputs, either from the input layer or from the outputs of neurons in an immediately preceding hidden layer. In general, each node may apply a function to its inputs to produce an output for that node. Nodes in hidden layers (e.g., learning layers) may apply the same function to their respective input(s) to produce their respective output(s). Some nodes, however, such as the nodes in the input layer InL receive only one input and may be passive, meaning that they simply relay the values of their single input to their output(s), e.g., they provide a copy of their input to their output(s), as illustratively shown by dotted arrows within the nodes of input layer InL.

For illustration purposes, FIG. 17 shows a simplified neural network consisting of an input layer InL′, a hidden layer HL1′, and an output layer OutL′. Input layer InL′ is shown having two input nodes i1 and i2 that respectively receive inputs Input_1 and Input_2 (e.g. the input nodes of layer InL′ receive an input vector of two dimensions). The input layer InL′ feeds forward to one hidden layer HL1′ having two nodes h1 and h2, which in turn feeds forward to an output layer OutL′ of two nodes o1 and o2. Interconnections, or links, between neurons (illustrative shown as solid arrows) have weights w1 to w8. Typically, except for the input layer, a node (neuron) may receive as input the outputs of nodes in its immediately preceding layer. Each node may calculate its output by multiplying each of its inputs by each input's corresponding interconnection weight, summing the products of it inputs, adding (or multiplying by) a constant defined by another weight or bias that may be associated with that particular node (e.g., node weights w9, w10, w11, w12 respectively corresponding to nodes h1, h2, o1, and o2), and then applying a non-linear function or logarithmic function to the result. The non-linear function may be termed an activation function or transfer function. Multiple activation functions are known the art, and selection of a specific activation function is not critical to the present discussion. It is noted, however, that operation of the ML model, or behavior of the neural net, is dependent upon weight values, which may be learned so that the neural network provides a desired output for a given input.

The neural net learns (e.g., is trained to determine) appropriate weight values to achieve a desired output for a given input during a training, or learning, stage. Before the neural net is trained, each weight may be individually assigned an initial (e.g., random and optionally non-zero) value, e.g., a random-number seed. Various methods of assigning initial weights are known in the art. The weights are then trained (optimized) so that for a given training vector input, the neural network produces an output close to a desired (predetermined) training vector output. For example, the weights may be incrementally adjusted in thousands of iterative cycles by a technique termed back-propagation. In each cycle of back-propagation, a training input (e.g., vector input or training input image/sample) is fed forward through the neural network to determine its actual output (e.g., vector output). An error for each output neuron, or output node, is then calculated based on the actual neuron output and a target training output for that neuron (e.g., a training output image/sample corresponding to the present training input image/sample). One then propagates back through the neural network (in a direction from the output layer back to the input layer) updating the weights based on how much effect each weight has on the overall error so that the output of the neural network moves closer to the desired training output. This cycle is then repeated until the actual output of the neural network is within an acceptable error range of the desired training output for the given training input. As it would be understood, each training input may include many back-propagation iterations before achieving a desired error range. Typically, an epoch refers to one back-propagation iteration (e.g., one forward pass and one backward pass) of all the training samples, such that training a neural network may include many epochs. Generally, the larger the training set, the better the performance of the trained ML model, so various data augmentation methods may be used to increase the size of the training set. For example, when the training set includes pairs of corresponding training input images and training output images, the training images may be divided into multiple corresponding image segments (or patches). Corresponding patches from a training input image and training output image may be paired to define multiple training patch pairs from one input/output image pair, which enlarges the training set. Training on large training sets, however, places high demands on computing resources, e.g., memory and data processing resources. Computing demands may be reduced by dividing a large training set into multiple mini-batches, where the mini-batch size defines the number of training samples in one forward/backward pass. In this case, and one epoch may include multiple mini-batches. Another issue is the possibility of a NN overfitting a training set such that its capacity to generalize from a specific input to a different input is reduced. Issues of overfitting may be mitigated by creating an ensemble of neural networks or by randomly dropping out nodes within a neural network during training, which effectively removes the dropped nodes from the neural network. Various dropout regulation methods, such as inverse dropout, are known in the art.

It is noted that the operation of a trained NN machine model is not a straight-forward algorithm of operational/analyzing steps. Indeed, when a trained NN machine model receives an input, the input is not analyzed in the traditional sense. Rather, irrespective of the subject or nature of the input (e.g., a vector defining a live image/scan or a vector defining some other entity, such as a demographic description or a record of activity) the input will be subjected to the same predefined architectural construct of the trained neural network (e.g., the same nodal/layer arrangement, trained weight and bias values, predefined convolution/deconvolution operations, activation functions, pooling operations, etc.), and it may not be clear how the trained network's architectural construct produces its output. Furthermore, the values of the trained weights and biases are not deterministic and depend upon many factors, such as the amount of time the neural network is given for training (e.g., the number of epochs in training), the random starting values of the weights before training starts, the computer architecture of the machine on which the NN is trained, selection of training samples, distribution of the training samples among multiple mini-batches, choice of activation function(s), choice of error function(s) that modify the weights, and even if training is interrupted on one machine (e.g., having a first computer architecture) and completed on another machine (e.g., having a different computer architecture). The point is that the reasons why a trained ML model reaches certain outputs is not clear, and much research is currently ongoing to attempt to determine the factors on which a ML model bases its outputs. Therefore, the processing of a neural network on live data cannot be reduced to a simple algorithm of steps. Rather, its operation is dependent upon its training architecture, training sample sets, training sequence, and various circumstances in the training of the ML model.

In summary, construction of a NN machine learning model may include a learning (or training) stage and a classification (or operational) stage. In the learning stage, the neural network may be trained for a specific purpose and may be provided with a set of training examples, including training (sample) inputs and training (sample) outputs, and optionally including a set of validation examples to test the progress of the training. During this learning process, various weights associated with nodes and node-interconnections in the neural network are incrementally adjusted in order to reduce an error between an actual output of the neural network and the desired training output. In this manner, a multi-layer feed-forward neural network (such as discussed above) may be made capable of approximating any measurable function to any desired degree of accuracy. The result of the learning stage is a (neural network) machine learning (ML) model that has been learned (e.g., trained). In the operational stage, a set of test inputs (or live inputs) may be submitted to the learned (trained) ML model, which may apply what it has learned to produce an output prediction based on the test inputs.

Like the regular neural networks of FIGS. 11 and 12, convolutional neural networks (CNN) are also made up of neurons that have learnable weights and biases. Each neuron receives inputs, performs an operation (e.g., dot product), and is optionally followed by a non-linearity. The CNN, however, may receive raw image pixels at one end (e.g., the input end) and provide classification (or class) scores at the other end (e.g., the output end). Because CNNs expect an image as input, they are optimized for working with volumes (e.g., pixel height and width of an image, plus the depth of the image, e.g., color depth such as an RGB depth defined of three colors: red, green, and blue). For example, the layers of a CNN may be optimized for neurons arranged in 3 dimensions. The neurons in a CNN layer may also be connected to a small region of the layer before it, instead of all of the neurons in a fully-connected NN. The final output layer of a CNN may reduce a full image into a single vector (classification) arranged along the depth dimension.

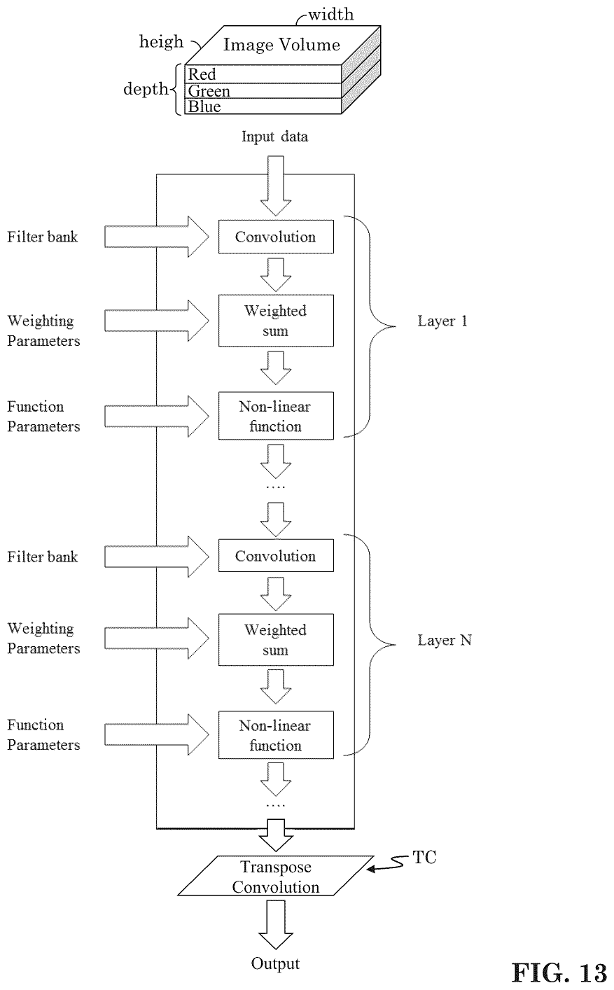

FIG. 13 provides an example convolutional neural network architecture. A convolutional neural network may be defined as a sequence of two or more layers (e.g., Layer 1 to Layer N), where a layer may include a (image) convolution step, a weighted sum (of results) step, and a non-linear function step. The convolution may be performed on its input data by applying a filter (or kernel), e.g., on a moving window across the input data, to produce a feature map. Each layer and component of a layer may have different pre-determined filters (from a filter bank), weights (or weighting parameters), and/or function parameters. In the present example, the input data is an image, which may be raw pixel values of the image, of a given pixel height and width. In the present example, the input image is illustrated as having a depth of three color channels RGB (Red, Green, and Blue). Optionally, the input image may undergo various preprocessing, and the preprocessing results may be input in place of, or in addition to, the raw input image. Some examples of image preprocessing may include: retina blood vessel map segmentation, color space conversion, adaptive histogram equalization, connected components generation, etc. Within a layer, a dot product may be computed between the given weights and a small region they are connected to in the input volume. Many ways of configuring a CNN are known in the art, but as an example, a layer may be configured to apply an elementwise activation function, such as max (0,x) thresholding at zero. A pooling function may be performed (e.g., along the x-y directions) to down-sample a volume. A fully-connected layer may be used to determine the classification output and produce a one-dimensional output vector, which has been found useful for image recognition and classification. However, for image segmentation, the CNN may classify each pixel. Since each CNN layers tends to reduce the resolution of the input image, another stage may be included to up-sample the image back to its original resolution. This may be achieved by application of a transpose convolution (or deconvolution) stage TC, which typically does not use any predefine interpolation method, and instead has learnable parameters.

Convolutional Neural Networks have been successfully applied to many computer vision problems. As explained above, training a CNN generally includes a large training dataset. The U-Net architecture is based on CNNs and can generally be trained on a smaller training dataset than conventional CNNs.

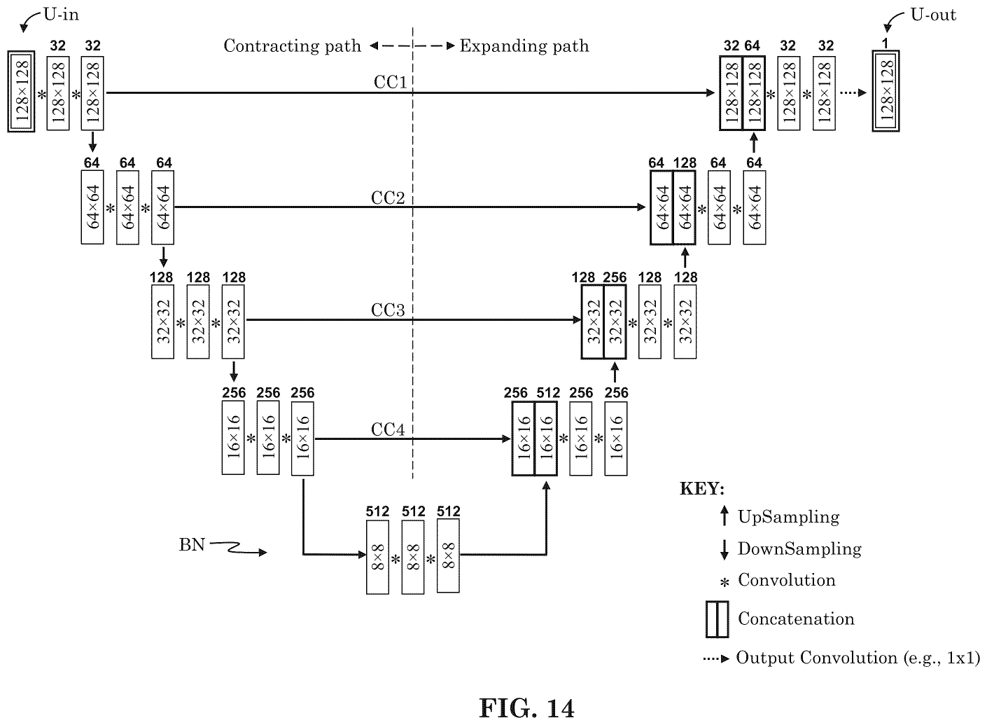

FIG. 14 illustrates an example U-Net architecture. The present exemplary U-Net includes an input module (or input layer or stage) that receives an input U-in (e.g., input image or image patch) of any given size. For illustration purposes, the image size at any stage, or layer, is indicated within a box that represents the image, e.g., the input module encloses number “128×128” to indicate that input image U-in is comprised of 128 by 128 pixels. The input image may be a fundus image, an OCT/OCTA enface, B-scan image, etc. It is to be understood, however, that the input may be of any size or dimension. For example, the input image may be an RGB color image, monochrome image, volume image, etc. The input image undergoes a series of processing layers, each of which is illustrated with exemplary sizes, but these sizes are illustration purposes only and would depend, for example, upon the size of the image, convolution filter, and/or pooling stages. The present architecture consists of a contracting path (herein illustratively comprised of four encoding modules) followed by an expanding path (herein illustratively comprised of four decoding modules), and copy-and-crop links (e.g., CC1 to CC4) between corresponding modules/stages that copy the output of one encoding module in the contracting path and concatenates it to (e.g., appends it to the back of) the up-converted input of a correspond decoding module in the expanding path. This results in a characteristic U-shape, from which the architecture draws its name. Optionally, such as for computational considerations, a “bottleneck” module/stage (BN) may be positioned between the contracting path and the expanding path. The bottleneck BN may consist of two convolutional layers (with batch normalization and optional dropout).

The contracting path is similar to an encoder, and generally captures context (or feature) information by the use of feature maps. In the present example, each encoding module in the contracting path may include two or more convolutional layers, illustratively indicated by an asterisk symbol “*”, and which may be followed by a max pooling layer (e.g., DownSampling layer). For example, input image U-in is illustratively shown to undergo two convolution layers, each with 32 feature maps. As it would be understood, each convolution kernel produces a feature map (e.g., the output from a convolution operation with a given kernel is an image typically termed a “feature map”). For example, input U-in undergoes a first convolution that applies 32 convolution kernels (not shown) to produce an output consisting of 32 respective feature maps. However, as it is known in the art, the number of feature maps produced by a convolution operation may be adjusted (up or down). For example, the number of feature maps may be reduced by averaging groups of feature maps, dropping some feature maps, or other known method of feature map reduction. In the present example, this first convolution is followed by a second convolution whose output is limited to 32 feature maps. Another way to envision feature maps may be to think of the output of a convolution layer as a 3D image whose 2D dimension is given by the listed X-Y planar pixel dimension (e.g., 128×128 pixels), and whose depth is given by the number of feature maps (e.g., 32 planar images deep). Following this analogy, the output of the second convolution (e.g., the output of the first encoding module in the contracting path) may be described as a 128×128×32 image. The output from the second convolution then undergoes a pooling operation, which reduces the 2D dimension of each feature map (e.g., the X and Y dimensions may each be reduced by half). The pooling operation may be embodied within the DownSampling operation, as indicated by a downward arrow. Several pooling methods, such as max pooling, are known in the art and the specific pooling method is not critical to the present disclosure. The number of feature maps may double at each pooling, starting with 32 feature maps in the first encoding module (or block), 64 in the second encoding module, and so on. The contracting path thus forms a convolutional network consisting of multiple encoding modules (or stages or blocks). As is typical of convolutional networks, each encoding module may provide at least one convolution stage followed by an activation function (e.g., a rectified linear unit (ReLU) or sigmoid layer), not shown, and a max pooling operation. Generally, an activation function introduces non-linearity into a layer (e.g., to help avoid overfitting issues), receives the results of a layer, and determines whether to “activate” the output (e.g., determines whether the value of a given node meets predefined criteria to have an output forwarded to a next layer/node). In summary, the contracting path generally reduces spatial information while increasing feature information.

The expanding path is similar to a decoder, and among other things, may provide localization and spatial information for the results of the contracting path, despite the down sampling and any max-pooling performed in the contracting stage. The expanding path includes multiple decoding modules, where each decoding module concatenates its current up-converted input with the output of a corresponding encoding module. In this manner, feature and spatial information are combined in the expanding path through a sequence of up-convolutions (e.g., UpSampling or transpose convolutions or deconvolutions) and concatenations with high-resolution features from the contracting path (e.g., via CC1 to CC4). Thus, the output of a deconvolution layer is concatenated with the corresponding (optionally cropped) feature map from the contracting path, followed by two convolutional layers and activation function (with optional batch normalization).

The output from the last expanding module in the expanding path may be fed to another processing/training block or layer, such as a classifier block, that may be trained along with the U-Net architecture. Alternatively, or in addition, the output of the last upsampling block (at the end of the expanding path) may be submitted to another convolution (e.g., an output convolution) operation, as indicated by a dotted arrow, before producing its output U-out. The kernel size of output convolution may be selected to reduce the dimensions of the last upsampling block to a desired size. For example, the neural network may have multiple features per pixels right before reaching the output convolution, which may provide a 1×1 convolution operation to combine these multiple features into a single output value per pixel, on a pixel-by-pixel level.

### Computing Device/System

FIG. 15 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the disclosure has been described in conjunction with several specific embodiments, it is evident to those skilled in the art that many further alternatives, modifications, and variations will be apparent in light of the foregoing description. Thus, the disclosure described herein is intended to embrace all such alternatives, modifications, applications and variations as may fall within the spirit and scope of the appended claims.

We claim:

1. A method for extracting structural retinal layer information of an eye from optical coherence tomography angiography (OCTA) volume data, comprising:
accessing a plurality of OCTA enface images from the OCTA volume data;
submitting the enface images to a data processing module configured to identify regions of superficial vascular plexus (SVP) and deep vascular plexus (DVP) within the enface images; and
designating at least one identified region of DVP that is superficial to an identified SVP region as a retinal layer.

2. The method of claim 1, wherein the data processing module includes a machine model whose training data set includes a combination of single class images and synthetic 2 class images, wherein:
the single class images are comprised of data from only superficial plexuses, deep plexuses, or avascular regions; and
the synthetic 2 class images are defined by pairing single class images and their corresponding masks including blending a randomly placed and sized region from an adjacent single class image into each synthetic 2 class image.

3. The method of claim 1, wherein the data processing module includes a deep learning machine model trained to identify and categorize vasculatures within OCTA data in the absence of OCT structural data.

4. The method of claim 1, wherein the at least one identified region of DVP that is superficial to an identified SVP region is designated a retinal nerve fiber layer (RNFL).

5. The method of claim 4, wherein the region designated as a retinal nerve fiber layer is incorporated into a retinal layer segmentation algorithm.

6. The method of claim 4, wherein the upper boundary of the designated RNFL is defined by the inner limiting membrane (ILM) of the eye.

7. The method of claim 1, wherein the at least one identified region of DVP is reclassified as a subdivision of the SVP.

8. A method for identifying a target tissue region in a volume of retinal tissue of an eye, comprising:
using a data processor,
accessing a plurality of OCTA enface images from OCTA volume data collected from the eye;
identifying superficial retina vessels within the eye based on the collected en face images; and
designating the area between the identified superficial retinal vessels and the inner limiting membrane (ILM) of the eye as the target tissue.

9. The method of claim 8, wherein identification of the inner limiting membrane is based on a its definition within an optical coherence tomography (OCT) scan.

10. The method of claim 8, wherein the data processor uses a machine learning model or conventional image processing to identify the area between the superficial retinal vessel and the inner limiting membrane.

11. The method of claim 8, further comprising incorporating the identified area between the ILM at the top and the superficial retinal vessels below into an automated retinal layer segmentation algorithm.
